(19) United States
(12) Patent Application Publication
(10) Pub. No.: US 2017/0240307 A1
(43) Pub. Date: Aug. 24, 2017

(54) PACKAGING STATION FOR ELASTOMERIC RINGS

(71) Applicant: Terence LaRocca, Ronkonkoma, NY (US)

(72) Inventors: Terence LaRocca, Selden, NY (US); Christopher J. Papson, Huntington Station, NY (US); Matt Tarry, Ronkonkoma, NY (US)

(21) Appl. No.: 15/434,910

(22) Filed: Feb. 16, 2017

Related U.S. Application Data
(60) Provisional application No. 62/297,665, filed on Feb. 19, 2016.

(51) Int. Cl. B65B 35/16; A61K 9/00; A61F 6/00; A61F 6/14; B65B 5/04; B65B 7/02

(52) U.S. Cl. CPC B65B 35/16; B65B 5/045; B65B 7/02; A61F 6/005; A61F 6/14; A61K 9/0036

(57) ABSTRACT

An automatic loader and packager station inserts and packages sensitive silicone vaginal rings into sealable pouches, wherein the silicone material is malleable, viscous and sticky, making it hard to accurately insert and seal the rings in a medically sealed package. The circular vaginal rings are axially elongated to reduce their width into the loading position, for accurate loading into scalable package pouches, without contamination of exterior openings of the respective package pouches into which the vaginal rings are inserted for sealed distribution. To accommodate malleable, viscous and/or sticky vaginal rings from manufacture to insertion into sealable pouches, the vaginal rings are moved upon a low friction, preferably roller-based conveyor before being loaded into the sealable pouches.

### RELATED APPLICATIONS

This application claims benefit and priority under 35USC§119 e) from provisional application Ser. No. 62/297,665 filed Feb. 19, 2016. The '665 application is incorporated by reference herein.

### FIELD OF THE INVENTION

The present invention relates to an automatic loader and packager of sensitive silicone vaginal rings, wherein the silicone material is malleable, viscous and sticky, making it hard to accurately insert and seal the rings in a medically sealed package.

### BACKGROUND OF THE INVENTION

Elastomeric rings, such as rubber O-rings, are used in a wide variety of industrial, commercial or consumer goods applications. Unless coated with lubricant which may be sticky, they have no special packaging requirements. Contrast that with the challenge of efficiently packaging vaginal or intravaginal elastomeric rings in a clean environment. These rings of elastomers such as silicone, EVA, or polyurethane are infused or coated with a wide variety of medicinal preparations for use as antibiotics, hormonal treatments, etc., including anti-HIV drugs for female patients. Since the medicinal components may be harmful to those not being treated, exposure to skin contact is to be avoided. Care must also be exercised to avoid any cross contamination. Two exemplary U.S. Patents regarding the make-up of these vaginal or intravaginal rings are U.S. Pat. No. 4,012,496 of Schopflin et al. and U.S. Pat. No. 8,580,294 of Malcolm et al.

According to Tong, in “A New Way to Prevent HIV Using Silicone Vaginal Rings”, in thebodypro.com website Sep. 21, 2015, researchers in France developed slow release of HIV treatment drugs in silicone vaginal rings over an extended period of time, According to both Szabo, in “Vaginal Silicone Ring Aids in HIV/Herpes Prevention”, in specialtypharmacytimes.com, Oct. 8, 2015, and Gibney, in “Silicone vaginal ring could deliver HIV, herpes drugs at low cost”, in fiercedrudelivery.com, Sep. 22, 2015, vaginal silicone rings can release hydrophilic molecules that are active against both HIV and herpes virus. The hydrophilic molecules include both tenofovir molecules active against HIV-1 and acyclovir molecules active against herpes virus, even though silicone is hydrophobic.

While the effectiveness, of silicone vaginal rings for the control of sexually transmitted infections (STI's) in developing countries among women has been established in several studies, a most attractive aspect is that these would be administered under the control of the women themselves, unlike the use of condoms which is controlled by the male population. A low cost method of packaging for the rings would enable their wide distribution complementing a low cost of manufacture of the elastomer rings.

### OBJECTS OF THE INVENTION

It is therefore an object of the present invention to accurately load and package malleable, viscous and sticky pharmaceutically and medically coated, or impregnated, silicone or other elastomeric vaginal rings, into sealable pouches for individual use for vaginal or intra-vaginal insertions.

It is also an object of the present invention to provide sealed individual use vaginal rings for contraceptive or anti-viral use.

It is yet another object of the present, invention to provide low cost sealed packaging of vaginal rings for widespread, low cost distribution to the general populace.

It is a further object of the present invention to axially elongate circular vaginal rings for accurate loading into sealable package pouches without contamination of exterior openings of the respective package pouches into which the vaginal rings are inserted for sealed distribution.

It is also an object of the present invention to provide moving conveyors for moving vaginal rings without having the vaginal rings becoming stuck or contaminated during movement.

Other objects will become apparent from the following description of the present invention.

### SUMMARY OF THE INVENTION

In keeping with these objects and others which may become apparent, the present invention is a packaging station which completed malleable, viscous and/or sticky vaginal rings from the manufacturing line and places them in individual pouches preferably of foil laminate; then it seals them and makes them available for boxing. To achieve this goal, a specialized loader is configured and interfaced with a packaging machine, preferably such as a modified but commercially available QuickPouch Vertical™ packaging machine produced by Adaptive Manufacturing Technologies of Ronkonkoma, N.Y. The QuickPouch Vertical™ machine is designed to be easily customized to accommodate a wide variety of pouch sizes and materials as well as manual and machine loaders. It forms the pouches from one or more side webs of material with internal custom-sized cavity dies with edge sealers, prints information on the pouch material, slits multiple pouches vertically, seals the tops of the pouches, and cuts the individual pouches horizontally before they fall onto an outfeed conveyor.

The path from the manufacturing line to the loader involves an in feed conveyor or other transporter, appropriately suited for moving malleable, viscous and/or sticky objects, such as, for example, an input ring conveyor that moves the vaginal rings in a singulated queue to either a manual or automated pick and place station that physically picks up rings from the input conveyor and inserts them one at a time into the loader atop the QuickPouch Vertical™. The rings can also be transported into the loader via a dispenser of any angle, such as, for example, in a tubular magazine or other in feed device.

To handle a variety of vaginal rings, some of which may be slightly tacky or sticky, the input ring transporter, may optionally be a conveyor, which is preferably a driven roller-top type conveyor of special design. The rollers arc driven by bearing friction from their driven shafts and can be easily stopped as resistance is encountered. Also, the output end of the conveyor narrows down into a single file of rings and stops the rings awaiting pickup from this end. This is done for gentle positive handling of the rings. A vibratory conveyor may not be reliable at conveying these elastomer rings with high friction or actual sticky surfaces.

The pick and place station may be a human operator using an expanding gripper, such as, for example, special pliers with expanding tips. When grasped, this expanding gripper tool engages an elastomeric ring from the end of the input transporter, such as a magazine or conveyor, and then stretches it somewhat to carry it to the input slot in the loader for deposit there. A pick and place robotic arm with an appropriate end effector which mimics the manual procedure could be used in higher volume applications.

The circular vaginal rings are axially expanded prior to insertion into a respective sealable package pouch, so that they are taller in vertical height than their width extending horizontally side to side when inserted into a loader above the respective open pouch. The leading edge of each vaginal ring is pushed downward into the pouch by a retractable finger, which has the effect of pulling each ring into its respective pouch, wherein the finger inserter arm of each pusher assembly is then retracted and the finger pusher assembly is raised up again to the loading position as is the chute assembly, which can have one or more chute assemblies.

At present the special elastomer ring loader as well as the QuickPouch Vertical™ machine units are powered, preferably being basically pneumatically operated as far as actuators are concerned. Compressed air pneumatic cylinders are used to “move things”. In future evolutions of this technology, the pneumatic components may be converted to electrically operated actuators; this may increase position accuracy and enhance throughput. Examples of substitute electric actuators are lead screws driven by servo or stepper motors as well as electromagnetic or piezoelectric linear motors.

While one or more silicone vaginal rings can be loaded simultaneously, in the present embodiment, the loader has preferably has two chute assemblies which accept a single elastomeric ring each while in the upper loading position. After both rings are inserted in the input groove areas sequentially, the chute assemblies are simultaneously lowered a small distance through the loading slot in the top panel of the QuickPouch Vertical™ machine, which preferably has formed the two pouches below. Then, as noted as aforesaid, the finger pusher assembly with pusher fingers extended engages the lower end of each stretched elastomer ring and pushes the rings down into the two side by side formed pouches where each ring falls free within each formed pouch. The finger of each pusher assembly is then retracted and the finger pusher assembly is raised up again to the loading position as is the dual chute assembly. The two loaded pouches are sealed at the top, cut apart, and both are dropped onto the outfeed conveyor. That is one complete packaging cycle. In other embodiments, any small number of adjacent chute assemblies can be implemented, for example from one to four. With more sophisticated pick and place robotic arms and compatible end effectors, the multiple chutes can be loaded simultaneously. Additionally, besides being transported vertically down in the one or more chute assemblies, the elongated elastomeric rings can be transported at any angle into pouches for packaging. Moreover, while a dual side buy side chute assembly is preferably used, it is anticipated that a single chute can be used, or there can he three or more chutes, oriented at any angle between horizontal or vertical.

It is further noted that while the drawings below show the loading assembly loading elastomeric silicone rings into the prior art QuickPouch Vertical™ machine, it is known that the loading assembly can be adapted to also load elastomeric silicone rings into other functionally equivalent or commercially available, pouch loading machines.

### BRIEF DESCRIPTION OF THE DRAWINGS

The present invention can best be understood, in connection with the accompanying drawings. It is noted that the invention is not limited to the precise embodiments shown in drawings, in which:

FIG. 1 is a perspective view of the packaging station for elastomeric rings of this invention;

FIG. 2 is a schematic representation of the movement of an elastomeric ring from manufacturing line to sealed package in an output bin; showing one or more horizontal in feed conveyors and slanted out put conveyors.

FIG. 2A is a schematic representation of an alternate embodiment for the movement of an elastomeric ring from manufacturing line to sealed package in an output bin, showing one or more vertical in feed magazine or other dispensers.

FIG. 3 is a front elevation of the packaging station of FIG. 1;

FIG. 4 is a side elevation of the packaging station of FIG. 1;

FIG. 5 is a top view of one embodiment for an input transporter, such as an input ring conveyor with linear output section;

FIG. 6 is a side detail of the input ring conveyor of FIG. 5, showing elastomeric rings atop friction driven rollers with belt driven shafts:

FIG. 7 is a perspective view of a simple end effector for a pick and place robotic arm which stretches the elastomer rings as it picks them up;

FIG. 7A is a diagrammatic top view of an elastomeric ring being stretched;

FIGS. 8, 8A, 9 and 9A are respective top plan, and side views showing the operation of an, expander tool for manual pickup of elastomeric rings stretching the rings as they are picked up;

FIG. 10 is a perspective exploded, view of one embodiment of various parts of the elastomer ring loader of this invention;

FIG. 11 is a front elevation of the a chute assembly, showing optionally a dual chute assembly;

FIG. 12 is a perspective view of an embodiment for a finger pusher assembly;

FIG. 13 is a detail of the pusher finger in the deployed position with a phantom view of an engaged elastomer ring;

FIG. 14 is a detail of the pusher finger in the retracted position ready to be raised to loading position, taken along dashed circle view line “14” of FIG. 12;

FIG. 15 is a perspective view of one embodiment for a chute assembly in the up loading position with the finger pusher assembly raised; and,

FIG. 16 is a side perspective view of the chute assembly partially lowered through the loading slot of the top panel of the QuickPouch Vertical™ machine.

### DETAILED DESCRIPTION OF THE INVENTION

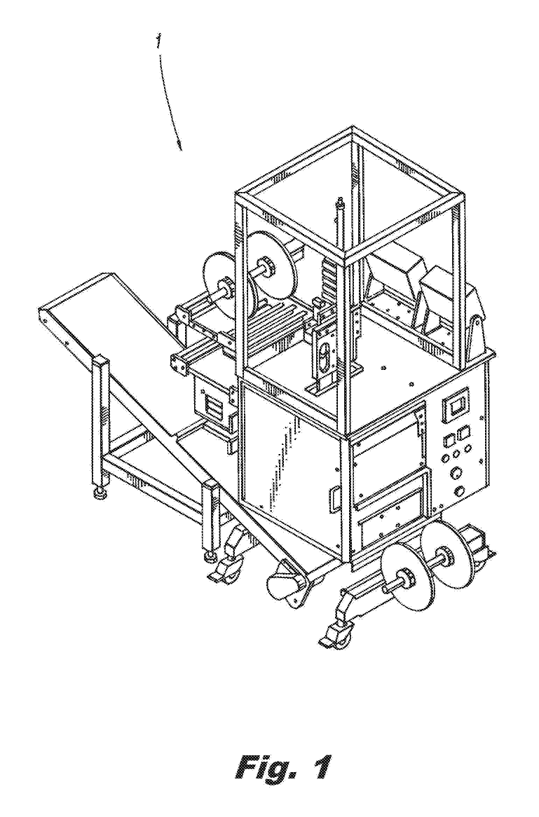

It is noted that the invention is not limited to the precise embodiments shown in the drawing FIGS. 1-16.

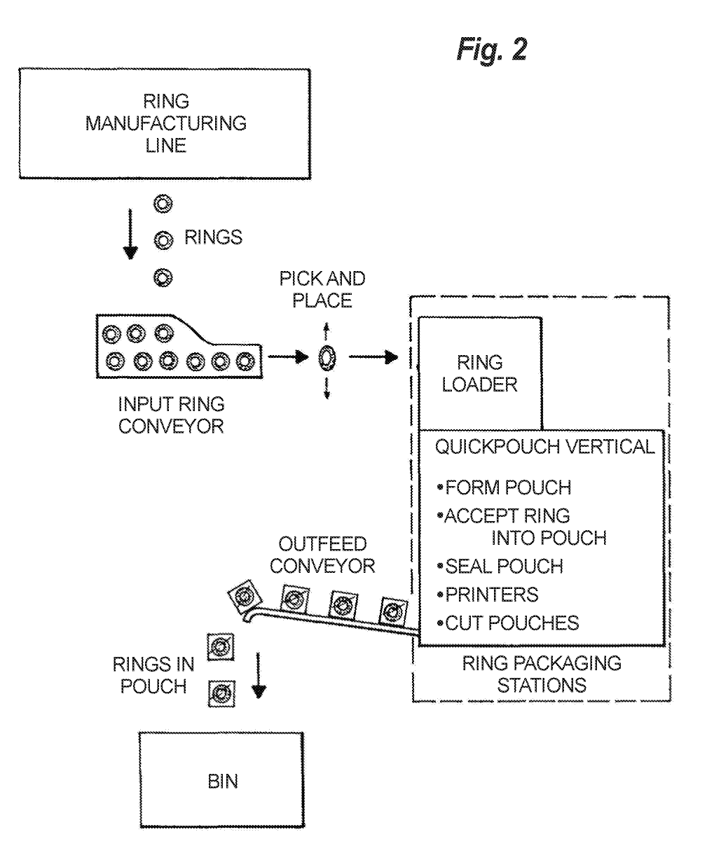

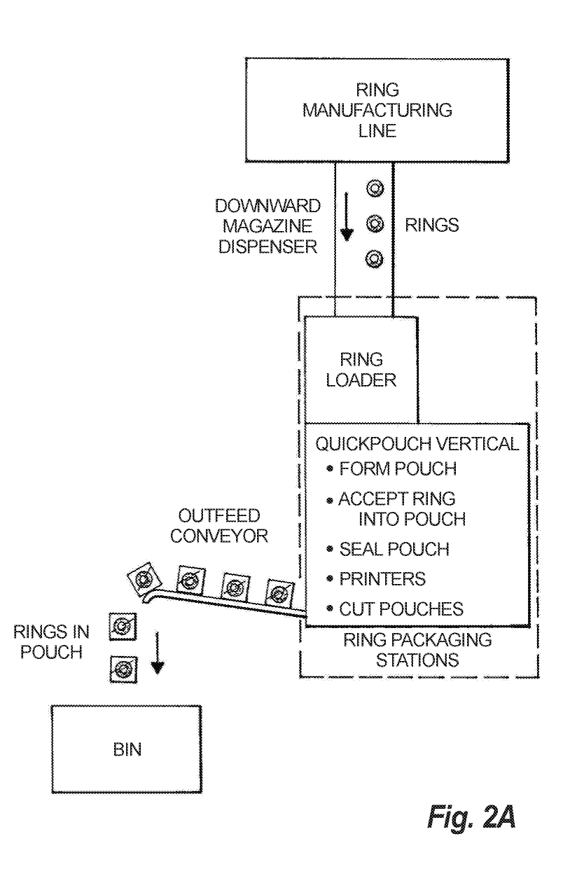

For example, FIG. 1 shows a view of one embodiment for the packaging station for elastomeric rings 15 of this invention. In the flow diagram of FIG. 2, the elastomeric ring manufacturing line outputs completed parts onto an input transporter, such as, for example, an input ring conveyor, from which a pick and place entity, such as a human user or a robotic arm, picks rings by stretching them onto a tool or end effector and deposits them serially into the ring loader which is attached to the top surface of the QuickPouch Vertical™ machine. The QuickPouch Vertical™ machine then completes the packaging function by placing each manufactured ring within a sealed pouch. This is done efficiently in a clean environment.

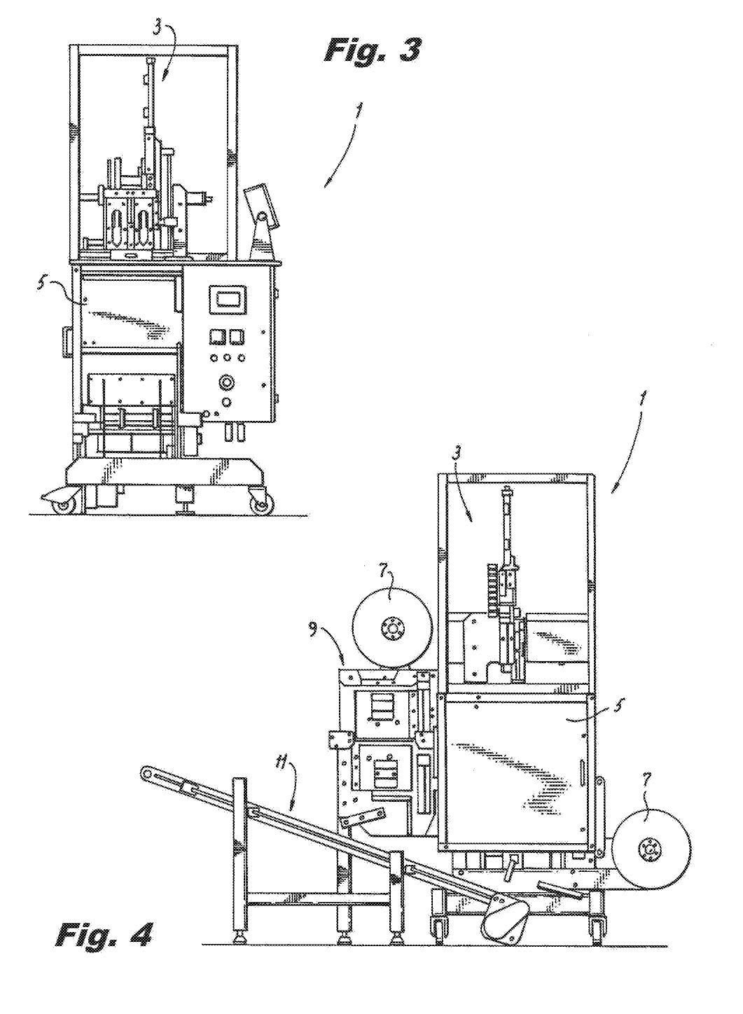

FIGS. 3 and 4 show a front and side view respectively of the packaging station 1 of this invention. Here can be seen the special elastomer ring loader 3 attached to the top of the QuickPouch Vertical™ machine 5. In FIG. 4 can be seen the advancing pouch forming material, with one or more web reels, such as, for example, the two web reels 7 which hold laminated foil material in dual pouch width. The dual printer, assembly 9 is used to annotate one side of each pouch. The completed filled and sealed pouches drop down onto the outfeed conveyor. While FIGS. 3 and 4 show the ring loader to be oriented vertically downward, it is known that the elastomeric rings can be optionally instead loaded horizontally, or at any angle between vertically and horizontally.

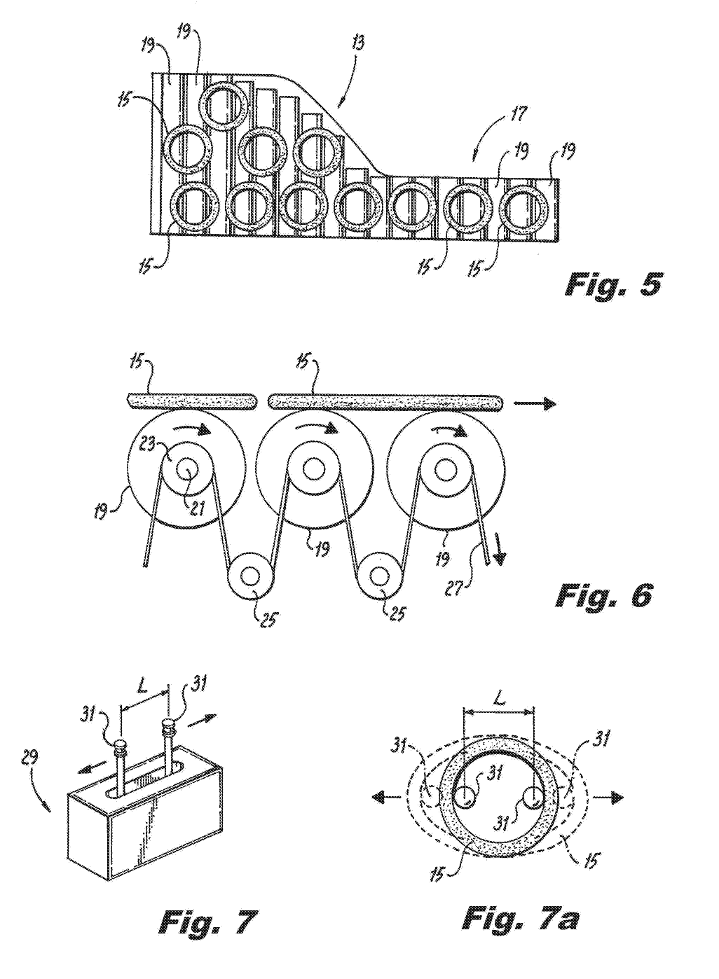

FIG. 5 shows a top view of an input conveyor, such as, for example, the roller-based input feed conveyor 13, which acts as a staging unit for the pick and place station which follows. It is noted that the elastomeric rings 15 lie flat atop rollers 19 and are gently conveyed to the narrowed end region 17 in a single file. In the detail of FIG. 6, pulleys 23 are driven in synchronism by belt 27; pulleys 23 are attached to roller shafts 21. Idler pulleys 25 preferably provide the required belt direction reversal. It is also noted that rollers 19 preferably are not attached to shafts 21, but they rotate by virtue of friction coupling to shafts 21. The inertia of rollers 19 as well as the bearing to shaft friction are carefully selected to provide a gentle drift to elastomeric rings 15 and the ability for the rings 15 to stop the rollers 19 if jammed or intentionally stopped such as at the output end. While the transporter is shown as having a horizontally oriented conveyor 13 in FIGS. 2, 5 and 6, it is known that other transporters, such as vertically oriented tubular magazines, such as shown in FIG. 2A, can be employed.

FIG. 7 shows a an alternate embodiment for the manually operable handler, such as the vaginal ring handler, which may be a simple end effector 29 for a robotic arm that will pick rings 15 from conveyor 13 by virtue of expanding the distance between pick rods 31 from U to a greater distance that will stretch rings 15 into the configuration shown in dashed lines. FIG. 7A shows the elastomeric ring 15 being stretched and elongated, with the robotic effector of FIG. 7.

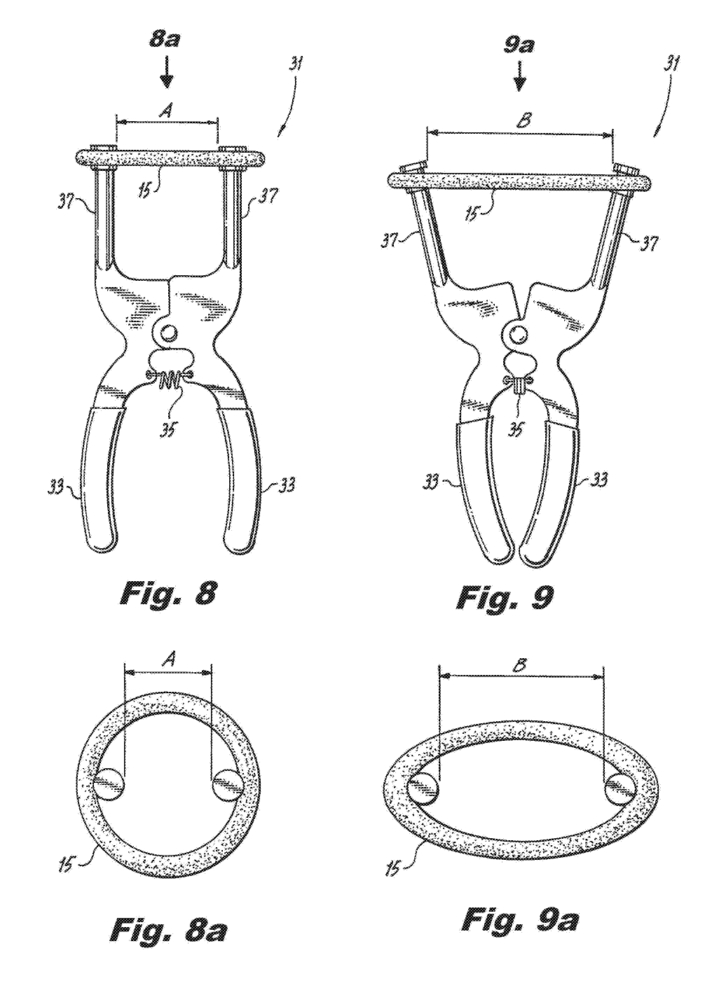

FIGS. 8 and 9 show an expander tool 31 to be used by an operator if the pick and place is handled manually. Compression spring 35 biases tool 31 to the closed position. Movable arms, such as squeezing handles 33 together causes engagement arms 37 to move farther apart. Elastomeric rings 15 are engaged at configuration “A” and handled in stretched configuration “B” to be inserted into ring loader 3.

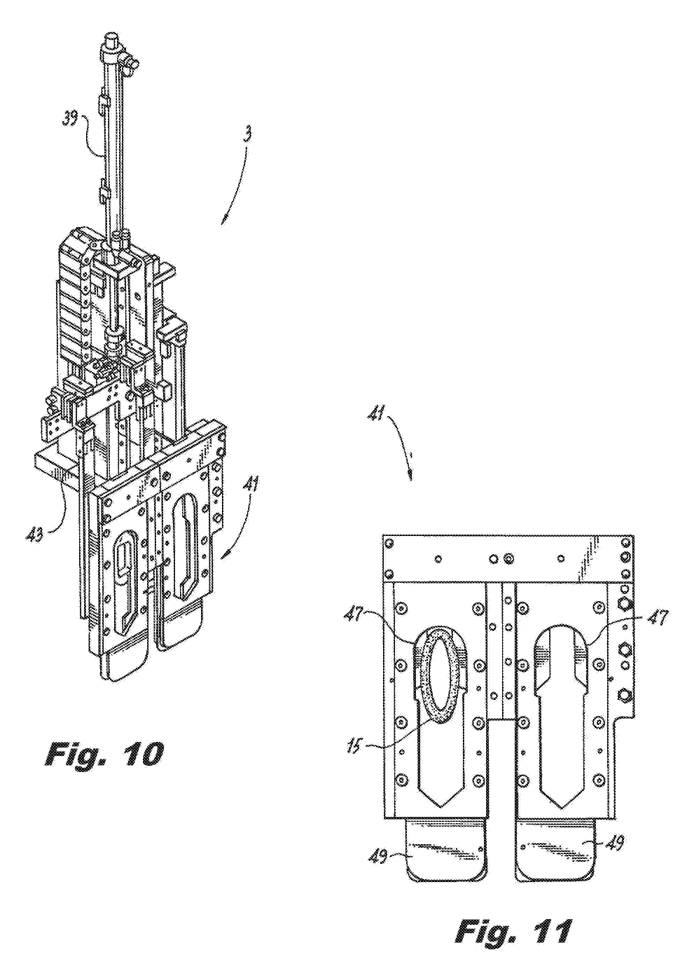

Elastomeric ring loader 3 is shown in FIG. 10. It preferably has insert assembly 39 powered by a pneumatic cylinder, chute assembly 41 to accommodate two elastomeric rings 15, and finger pusher assemblies 43 with one for each chute loading slot. FIG. 11 shows more details of the dual chute assembly 41. Rings 15 are loaded sequentially into load slots 47 as shown; when released they are retained in side grooves. Extensions 49 at the bottom insure that the rings 15 are properly guided between the flat sides of the pouch within the QuickPouch Vertical™ machine. While FIGS. 10 and 11 show preferably a pair of vertically oriented elastomeric ring loader chutes, other horizontal or other angled loader chutes can be used, and the loaders can have one, two or three or more chutes or other magazine type loaders, of any angled orientation, for one or more elastomeric rings 15.

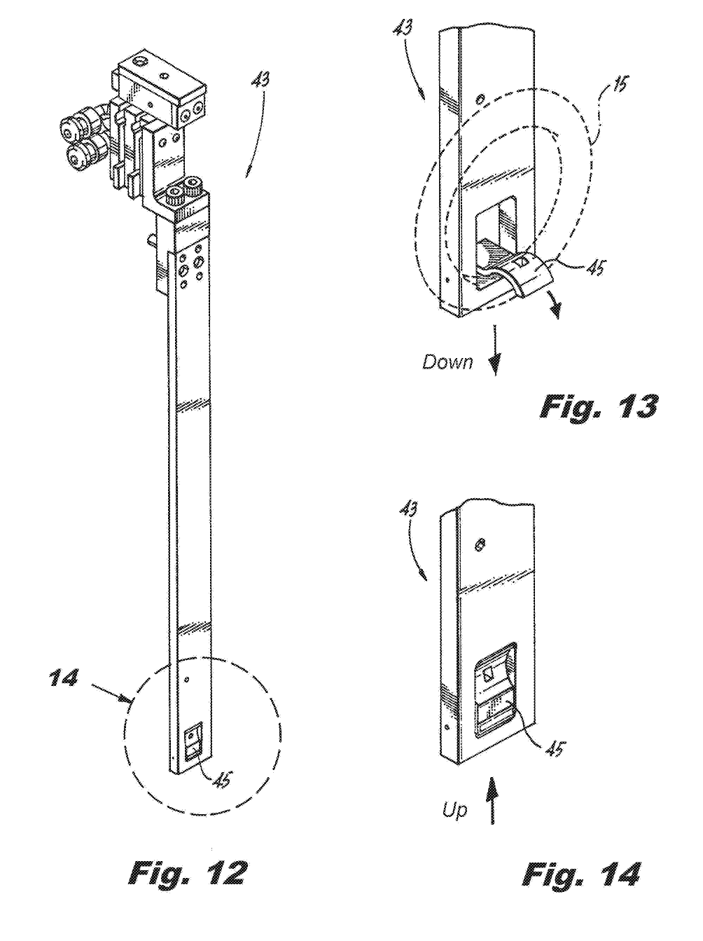

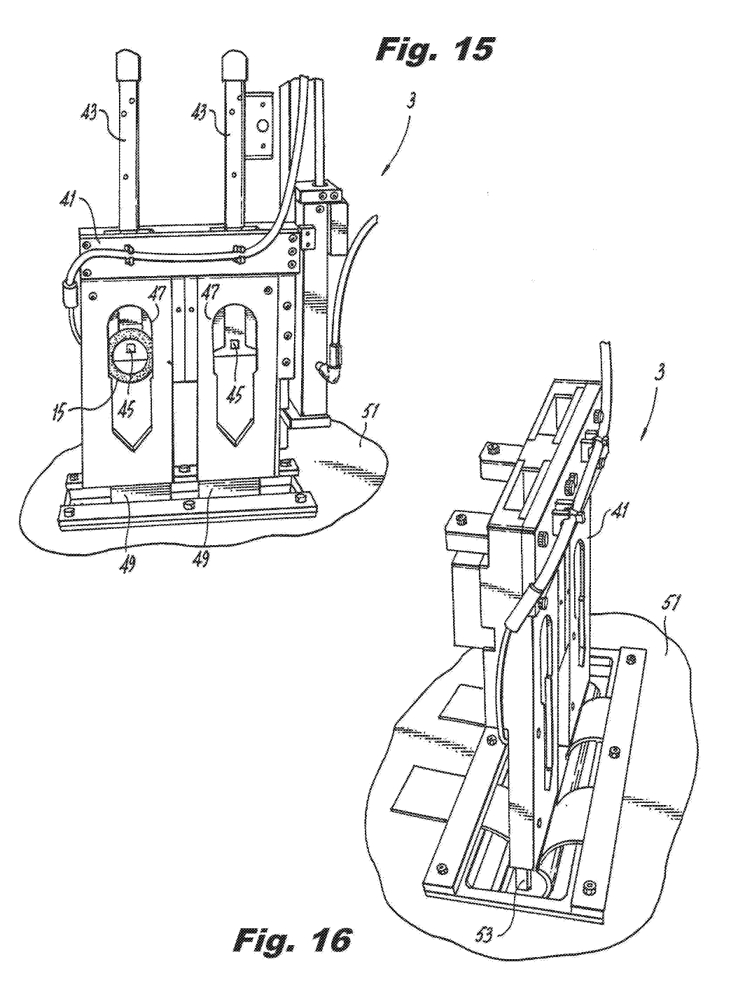

The finger pusher assembly 43 of FIG. 12 (one for each loading slot) is used to move both rings 15 from the loading slots 47 into the pouches underneath simultaneously. This is accomplished by deploying finger inserter arm 45 (see FIG. 13 detail) which engages the inner lower end of stretched ring 15. After the loading push down into the pouch, finger inserter arm 45 is retracted as shown in FIG. 14 before assembly 43 is again raised to avoid tearing of the pouch side. FIG. 15 shows finger pusher assembly 43 as well as dual chute assembly 41 in their uppermost loading position. It s noted that the bottom of extensions 49 are just at the level of the top 51 of the QuickPouch Vertical™ machine. Also noted is the position of finger 45 relative to the position of ring 15. Note that FIG. 16 shows dual chute assembly 41 pushed down so that extensions 49 are below top surface 51 extending into the loading slot 53.

It is further noted that while the drawings show the loading assembly loading elastomeric silicone rings 15 into the Quick Pouch Vertical machine 5, it is known that the loading assembly identified as elastomer ring loader 3, shown in FIGS. 3, 4, 10, 11, 12, 13, 14, 15 and 16 herein, can be adapted to also load elastomeric silicone rings 15 into other functionally equivalent or commercially available pouch loading machines, manually or, robotically, including, but not limited to machines, copies of some of which are attached in the Appendix to the specification herein.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention.

It is further known that other modifications may be made to the present invention, without departing the scope of the invention.

We claim:

1. A method of inserting and sealing a loaded elastomer ring in a package comprising the steps of:
transporting a circular, loaded elastomer ring to a pick and place station for insertion into a package having an opening;
using a tool for elongating said ring forming narrowed leading edges at opposite ends of said elongated ring;
moving said elongated ring into a loading slot with narrowed sides;
releasing and removing said tool from said ring, said narrowed sides retaining said ring in an elongated state;
using a retractable finger assembly to push a leading edge of said elongated ring out of said loading slot through said opening into said package;
retracting said finger assembly from said elongated ring, allowing said ring to return to its normal circular shape completely within said package; and
closing said opening into said package for providing a sealed pouch containing said loaded ring for distribution and ultimate use.

2. The method of claim 1 in which said loaded elastomer ring comprises a vaginal ring infused or coated with a medical composition selected from the group consisting of antibiotics, hormones, and anti-HIV drugs.

3. The method of claim 1 in which said tool comprises engagement arms actuated by squeezing a pair of handles overcoming a spring which biases said tool in a closed position to spread said engagement arms to stretch said ring to form said elongated ring with aid narrowed leading edges at opposite sides of said elongated ring.

4. The method of claim 1 in which said tool comprises a robotic arm.

5. The method of claim 1 in which said tool is used to move said elongated ring into said loading slot.

6. The method of claim 1 in which said retractable finger assembly comprises an, extended inserter arm with a finger opening located adjacent a distal end thereof, and a pivoted finger within said opening, said finger being pivoted out of said opening to engage and push said ring into said opening into said package, and being pivoted back into said opening to allow said extended inserter arm to be retracted from said package and avoid tearing of a side of said package, and to allow the inserter arm to be retracted without removing previously inserted rings.

7. The method of claim 1 in which multiple circular, loaded elastomer rings are transported sequentially to said pick and place station, said elongated elastomer rings are loaded into side by side loading slots, using multiple side by side retractable finger assemblies to insert said rings into openings of side by side packages followed by withdrawing said finger assemblies, and sealing said side by side packages to form multiple sealed pouches for packaging. shipping and distribution for customer use.

8. The method of claim 7 in which said loading slots are oriented with exit openings located at edges of said loading slots and said openings into said packages are located directly adjacent said exit openings of said loading slots to facilitate insertion of said rings into said packages,

9. Apparatus for inserting and sealing a loaded easterner ring in a package comprising:
a conveyor for transporting said elastomer ring to a pick and place station;
a tool for elongating said ring forming narrowed leading edges at opposite ends of said elongated ring and moving said elongated ring into a loading slot with narrowed sides, said tool being released to remove said tool from said ring, said narrowed sides retaining said ring in an elongated state;
a retractable finger assembly for pushing a leading edge of said elongated ring out of said loading slot through said opening into said package, said finger assembly being removed from said elongated ring, allowing said ring to return to its normal circular shape completely within said package; and
means for closing and sealing said opening into said package for providing a sealed pouch containing said loaded ring for distribution and ultimate use.

10. The apparatus of claim 9 in which said loaded elastomer ring comprises a vaginal ring infused or coated with a medical composition selected from the group consisting of antibiotics, hormones, and anti-HIV drugs.

11. The apparatus of claim 9 in which said tool comprises engagement arms actuated by squeezing a pair of handles overcoming a spring which biases said tool in a closed position to spread said engagement arms to stretch said ring to form said elongated ring with said narrowed leading edges at opposite sides of said elongated ring.

12. The apparatus of claim 9 in which said tool comprises a robotic arm.

13. The apparatus of claim 9 in which said retractable finger assembly comprises an extended inserter arm, with a finger opening located adjacent a distal end thereof, and a pivoted finger within said opening, said finger being pivoted out of said opening to engage and push said ring into said opening into said package, and being pivoted back into said opening to allow said extended inserter arm to be retracted from said package and avoid tearing of a side of said package, and to allow the inserter arm to be retracted without removing previously inserted rings.

14. The apparatus of claim 9 in which multiple circular, loaded elastomer rings are transported sequentially on said conveyor to said pick and place station, said circular, loaded elongated elastomer rings being loaded into side by side loading slits, and having multiple retractable finger assemblies to insert said rings into openings of side by side packages followed by withdrawing said finger assemblies, and means sealing said side by side packages to form multiple sealed pouches.

15. An elastomeric ring loader comprising:
an insert assembly comprising a chute assembly for accommodating multiple elongated, normally circular, elastomeric rings restrained in at least one chute loading slot;
a retractable finger assembly for said at least one loading slot for pushing a leading edge of each said elongated ring out of said at least one loading slot through an open end of a package; and
each said finger assembly comprising an extended inserter arm with a finger opening located adjacent a distal end thereof, and a pivoted finger within said finger opening, said finger being pivoted out of said finger opening to engage and push said ring from said chute loading slot into said opening into said package, and said finger being adapted to pivot back into said finger opening to allow said ring to return to its normal circular shape within said package, said extended inserter arm thereby able to be retracted from <said package and avoid tearing of a side of said package, and to allow the inserter arm to be retracted without removing previously inserted rings.

16. The elastomeric ring loader as in claim 15, wherein said at least one loading slot is a plurality of loading slots of a plurality of loading chutes.

17. The elastomeric ring loader of claim 15 in which said elastomer ring comprises a vaginal ring infused or coated with a medical composition selected from the group consisting of antibiotics, hormones, and anti-HIV drugs.

18. The elastomeric ring, loader of claim 17 in which said loading slots are vertically oriented with exit openings located at bottom edges of said loading slots.

19. The elastomeric ring loader of claim 18 in which openings into said packages are located directly under said exit openings of said loading slots to facilitate insertion of said rings into said packages.
